(19) United States
(12) Patent Application Publication
(10) Pub. No.: US 2023/0065829 A1
(43) Pub. Date: Mar. 2, 2023

(54) MICRO CATHETER CALIPER

(71) Applicant: Accumed Radial Systems, LLC, Farmington Hills, MI (US)

(72) Inventor: Joseph R. Korotko, Livonia, MI (US)

(21) Appl. No.: 17/900,259

(22) Filed: Aug. 31, 2022

Related U.S. Application Data
(60) Provisional application No. 63/239,005, filed on Aug. 31, 2021.

(51) Int. Cl. A61M 25/04; A61M 25/10

(52) U.S. Cl. CPC A61M 25/04; A61M 25/10; A61B 17/12136

(57) ABSTRACT

An example catheter caliper according to the present disclosure includes a tube having a plurality of markers at predetermined intervals and a wire extending from the tube, wherein the catheter caliper is configured to be received into the vasculature of a patient. Other example catheter calipers and example methods of using the catheter caliper is also disclosed.

### CROSS-REFERENCE TO RELATED APPLICATIONS

This application claims priority to U.S. Provisional Application 63/239,005 filed on Aug. 31, 2021, which is hereby incorporated by reference herein in its entirety.

### BACKGROUND

Certain medical procedures may benefit from the ability to perform intravascular length measurements. For instance, catheterization procedures that address or treat artifacts inside blood vessels such as blockages, or perforations/dissections, may benefit from the ability to measure those artifacts. In addition, it is at times beneficial to map the vasculature of a patient such as by determining a distance between adjacent branches in a vessel.

### BRIEF DESCRIPTION OF THE DRAWINGS

FIG. 1 shows an example catheter caliper.

FIG. 2 shows another example catheter caliper.

FIG. 3 shows an example catheter assembly for use with the example catheter caliper of FIG. 2.

FIG. 4 shows an example catheter caliper in use.

FIG. 5 shows another example catheter caliper.

FIG. 6A shows the example catheter caliper of FIG. 5 in a starting position.

FIG. 6B shows the example catheter caliper of FIG. 5 in an extended position.

FIG. 7 shows another example catheter caliper.

FIG. 8A shows the example catheter caliper of FIG. 7 in a starting position.

FIG. 8B shows the example catheter caliper of FIG. 7 in an extended position.

FIG. 9A shows a portion of the example catheter caliper of FIG. 7.

FIG. 9B shows a portion of the example catheter caliper of FIG. 7.

### SUMMARY

An example catheter caliper assembly according to the present disclosure includes, among other possible things, a catheter caliper with a tube having a plurality of markers at predetermined intervals and a wire extending from the tube. The catheter caliper is defined between a distal end of the tube and a proximal end of the wire, and is configured to be received into the vasculature of a patient.

An example catheter caliper assembly according to the present disclosure includes, among other possible things, a first component including an outer tube and a first wire extending from the outer tube. The outer tube has a first marker. The first component is defined between a distal end of the outer tube and a proximal end of the first wire. A second component is configured to be received in the outer tube and is defined between a proximal end and a distal end. The distal end has a second marker. A ruler is arranged at a proximal end of one of the first and second components. The ruler has a plurality of markings each spaced apart by predetermined intervals. A pointer is arranged at a proximal end of the other of the first and second components. The pointer is configured to point to the markings on the ruler.

An example method of making intravascular measurements according to the present disclosure includes, among other possible things, inserting a catheter caliper into a vasculature of a patient to an artifact or feature to be measured. The catheter caliper includes a tube having a plurality of radiopaque markers each spaced apart by predetermined intervals, and a wire extending from the tube. The catheter caliper is defined between a distal end of the tube and a proximal end of the wire. The method also includes measuring the length of the artifact or feature by comparing the length of the artifact or feature to the markers on the tube.

An example method of making intravascular measurements according to the present disclosure includes, among other possible things, inserting a catheter caliper into a vasculature of a patient to a location near an artifact or feature to be measured. The catheter caliper includes a first component including an outer tube and a first wire extending from the outer tube. The outer tube has a first marker. The first component is defined between a distal end of the outer tube and a proximal end of the first wire. A second component is configured to be received in the outer tube and is defined between a proximal end and a distal end. The distal end has a second marker. A ruler is arranged at a proximal end of one of the first and second components. The ruler has a plurality of markings each spaced apart by predetermined intervals. A pointer is arranged at a proximal end of the other of the first and second components. The pointer is configured to point to the markings on the ruler. The method also includes positioning the distal end of the first and second components past the artifact or feature, and moving the pointer with respect to the ruler such that the first marker moves with respect to the second marker and the first marker is positioned at a first end of the artifact or feature and the second marker is positioned at a second end of the artifact or feature.

### DETAILED DESCRIPTION

FIG. 1 shows an example catheter caliper 10 according to an embodiment. The example catheter caliper 10 may be used during surgical procedures to estimate intravascular lengths. The catheter caliper 10 is an elongate tubular structure 12 having a distal end 14 and a proximal end 16. The catheter caliper 10 could be made of any known polymeric material, for example. The catheter caliper 10 includes a portion 22 that has a plurality of markers 18 arranged near the distal end 14. In other examples, the portion 22 having markers 18 may extend a longer length of the catheter caliper 10, or even the entire length of the catheter caliper 10. The markers 18 may be marker bands that extend about the circumference of the tubular structure 12, for example.

The markers 18 are spaced apart by a known interval 20 to form a ruler for measurements. In one example, the markers 18 are spaced by 5 or 10 mm. In the illustrated example, four markers 18 are shown. In other examples, there are between five and ten markers 18 arranged at the distal end 14, but more or fewer markers 18 may be used. The markers 18 are used to determine a length, for example. The markers could be printed, molded, or otherwise included in the catheter caliper 10. The markers 18 may be radio-opaque, so that they are visible using known fluoroscopic imaging. The markers 18 may be made of platinum-based or iridium-based materials, for example, for visibility in imaging.

The catheter caliper 10 may be sized to have a diameter such that it can be used in the coronary arteries of the heart. The catheter caliper 10 may be any type of catheter, such as a guide catheter, for example. Although the catheter caliper 10 is shown to have a straight proximal end 16, other types of catheter ends could be used.

The catheter caliper may be used during an angioplasty procedure to determine the length of an artifact such as a blockage, perforation, or dissection in the vasculature of a patient in order to select the appropriate balloon catheter and stent, for example. An angioplasty procedure is used to repair a blockage, perforation, or dissection in a vessel. An angioplasty procedure typically involves a guide catheter and a balloon catheter (the balloon is used to repair the blockage in various ways, as would be known in the art). During the procedure, a guidewire may be inserted into the vessel via the access point and advanced towards the blockage or perforation/dissection. The catheter caliper 10 is inserted into the vessel via the access point and advanced towards the blockage or perforation/dissection along the guidewire. When the radiopaque markers 18 of the catheter caliper 10 are near the blockage or perforation/dissection, the physician or user can estimate the length of the blockage or perforation/dissection by comparing the length to the markers 18 having known spacing. The number of markers 18 that the blockage spans can then be used to determine a length measurement of the blockage or perforation/dissection. The length measurement can be used to select an appropriately sized stent for the repair.

Other applications for the catheter caliper are also contemplated. For instance, one may want to measure a feature of a patient's vasculature such as the distance between adjacent branches in the vasculature to select an appropriately sized stent, and place the stent in an appropriate location, without blocking off the branches. Additionally, other anatomical distances or locations could be measured with the catheter caliper, and that would benefit a medical procedure. It should be understood that the example applications described herein are non-limiting.

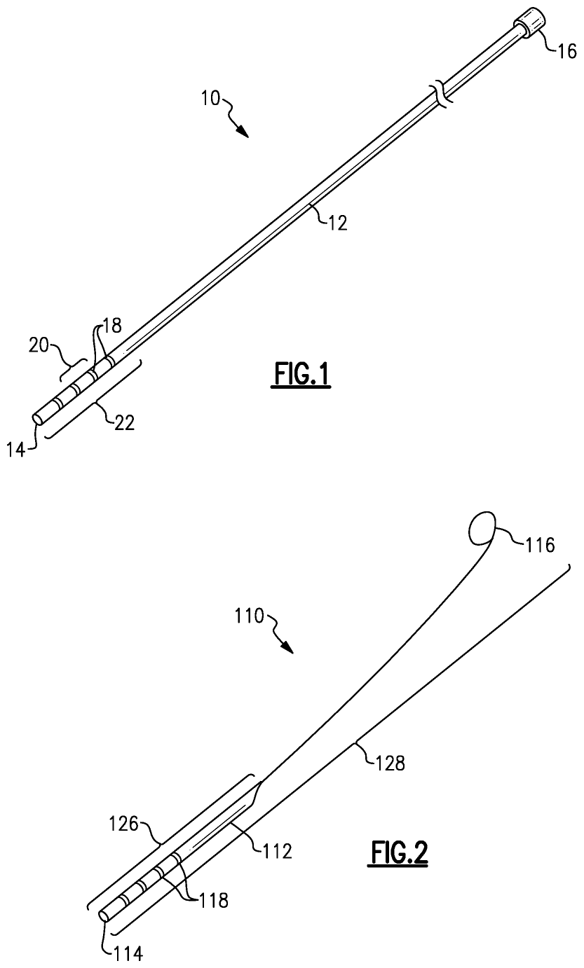

FIG. 2 shows another example catheter caliper according to an embodiment. To the extent not otherwise described or shown, the catheter caliper 110 corresponds to the catheter caliper 10 of FIG. 1, with like parts having reference numerals preappended with a “1.” In this example, the catheter caliper 110 has a tubular structure or portion 112 that is shorter in length than a full catheter. This example may be referred to as a “rapid exchange” or “Rx” catheter caliper. The tubular structure 112 is attached to a tether or wire 124. The tubular structure 112 may have a length 126 that is much shorter than the length 128 of the catheter caliper 110. Although four markers 118 are shown, more or fewer may be used. In one example, there may be markers 118 along most or all of the length 126 of the tubular portion 112. This example catheter caliper 110 having a short tubular structure 112 may be easier to insert and remove than a full catheter in some instances and may enable use with a shorter length guidewire.

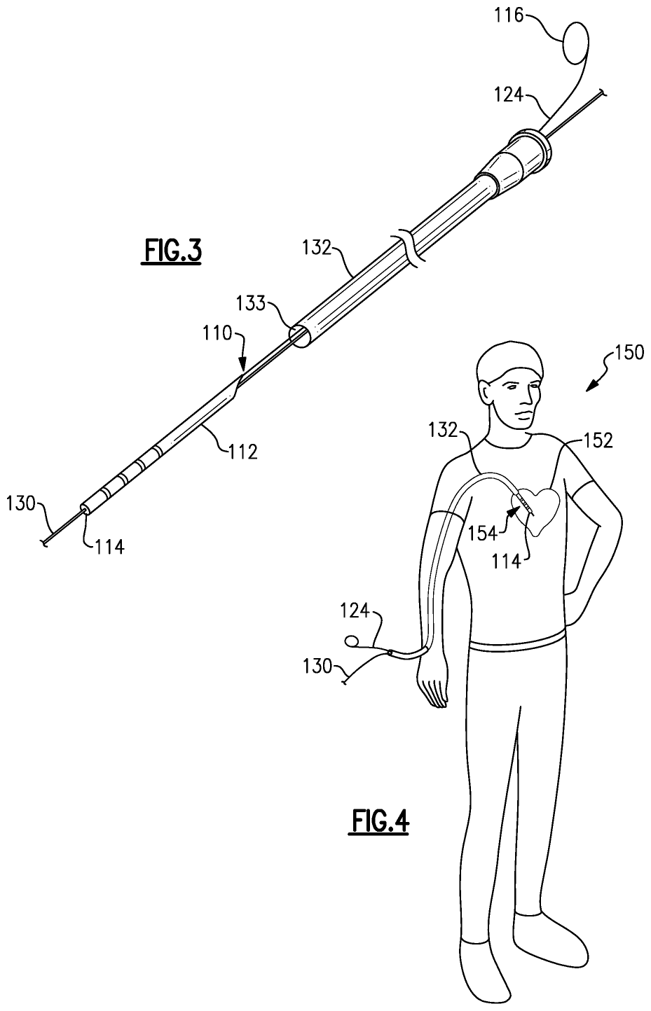

FIG. 3 shows an example catheter assembly for use with the example catheter caliper of FIG. 2. The catheter caliper 110 may be used with a guidewire 130 and guide catheter 132. The catheter caliper 110 is a “Rapid Exchange” or “Rx” catheter, as the guidewire 130 extends through the tubular portion 112 of the catheter caliper 110. The guidewire 130 and catheter caliper 110 are received inside the lumen 133 of the guide catheter 132. Although a guidewire 130, catheter caliper 110, and guide catheter 132 are shown, the catheter caliper 110 may be used with another combination of components. For example, the catheter caliper 10 of FIG. 1 may be configured as a guide catheter, and thus not be used with a separate guide catheter 132. In other embodiments, a plurality of spaced markers 118 may be on other components for making intravessel measurements. For example, there may be a plurality of markers on a guidewire 130, a guide catheter 132, a balloon catheter, or other component.

FIG. 4 shows an example catheter caliper in use. In this example, the catheter caliper 110 is used in a heart procedure. A physician or user could access the vascular arteries 152 of a patient 150 through an access point 148. In the illustrated example, the access point 148 is the radial artery at the patient's wrist. In other examples, the access point could be the femoral artery at the patient's groin. A guide catheter 132 is inserted into the patient 150 from the access point 148. A guidewire 130 is then inserted into the lumen 133 of the guide catheter 132 and advanced beyond the distal end of the guide catheter 132 into the coronary artery. The guidewire 130 may be advanced into a distal segment of the artery, such that a tip of the guidewire 130 is beyond the blockage or perforation/dissection 154. The catheter caliper 110 is then inserted over the guidewire 130 and within the lumen 133 of the guide catheter 132. The catheter caliper 110 is advanced along the guidewire 130 until the markers 118 are alongside the blockage or perforation/dissection 154. The physician or user can then make a length measurement of the blockage or perforation/dissection 154 by viewing the markers 118 via fluoroscopic imaging. Although fluoroscopic imaging is discussed, the guidewire and/or catheters may be monitored by any imaging technique known in the art. The physician or user can then choose the appropriate length angioplasty balloon and stent for the angioplasty procedure.

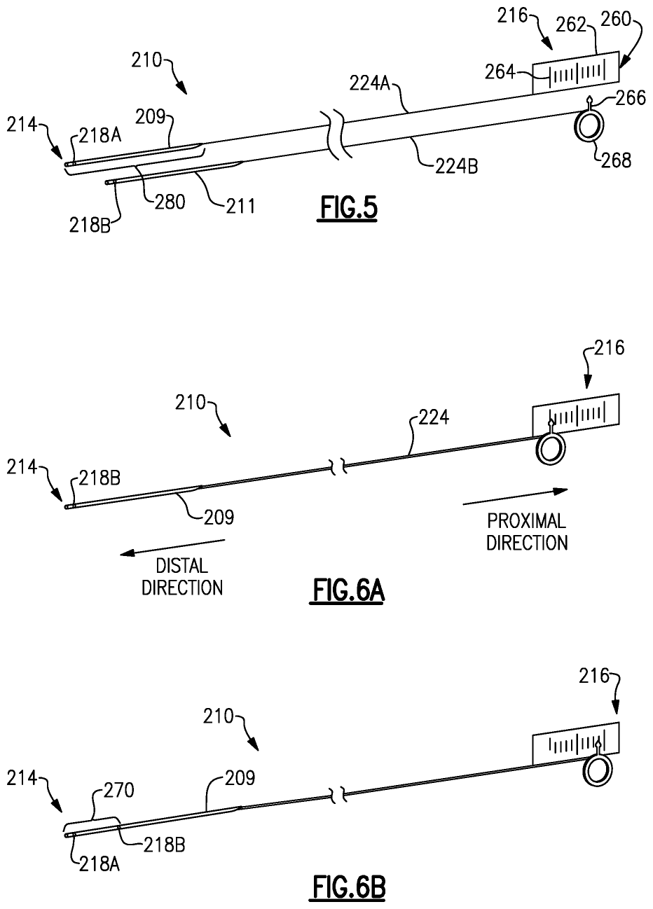

FIG. 5 shows another example catheter caliper. In this example, the catheter caliper 210 is a two-piece device, with the pieces shown unassembled to reveal the components, details and features. To the extent not otherwise described or shown, the catheter 210 corresponds to the catheter calipers 10, 110 of FIGS. 1-4, with like parts having reference numerals preappended with a “2.” The catheter caliper 210 has an outer tube 209 and an inner tube 211. In this view, the catheter caliper 210 is disassembled. The inner tube 211 is sized to fit within the outer tube 209 such that they are co-axial when assembled. The outer and inner tubes 209, 211 are connected to a proximal end 216 of the catheter caliper 210 via a wires 224A, 224B, respectively. The outer and inner tubes 209, 211 may then both fit within a guide catheter 132 and over a guidewire 130, for example. The outer tube 209 has a marker 218A and the inner tube 211 has a marker 218B. The markers 218A, 218B are near the distal end 214 of the tubes 209, 211. A scale 260 is arranged at the proximal end 216. The scale 260 generally includes a ruler 262 and a pointer 266. The ruler 262 has a plurality of spaced markers 264. The scale 260 may be a millimeter scale, for example, with the spaced markers 264 spaced apart by 1 or 5 millimeters. The pointer 266 may include a finger loop 268 or other handle, in some embodiments. The pointer 266 slides relative to the ruler 262 to indicate length measurements. A physician or other user may slide the pointer 266 along the scale 260 using the finger loop 268. As the pointer 266 is moved relative to the scale 260, the marker 218A moves relative to the marker 218B. In this example, the inner and outer tubes 209, 211 are “rapid exchange” catheters.

Although the pointer 266 is shown on the outer tube 209 and the ruler 262 is on the inner tube 211, these could be switched, in other examples. Although the tubes 209, 211 are connected to the scale 260 via wires 224A, 224B, in other examples, the scale 260 may be attached directly to the tubes 209, 211. In other words, the tubes 209, 211 could be an over-the-wire catheter with a longer tube.

The catheter caliper 210 may be a micro sized catheter, which enables it to be used in the coronary arteries of the heart and may be inserted into the diameter of an artery that is reduced smaller by a blockage. The catheter caliper 210 may be designed to interface with a coronary guidewire and coronary catheter. The inner tube 211 has an inner diameter that is big enough to receive a guidewire 130. A typical coronary guidewire 130 may have a diameter of about 0.014 inches and a typical guide catheter may have an inside diameter of about 0.040 inches. In one example, the inside diameter of the inner tube 211 has a diameter of about 0.016 inches and the outer diameter of the outer tube 209 may be about 0.038 inches to accommodate the guidewire and guide catheter. The tubes 209, 211 may have a length 280 that is much smaller than a total length of the catheter caliper 210. In one example, the length 280 may be between about 8 and 12 inches, while the total length of the catheter caliper 210 may be around 55 inches, for example. The catheter caliper 210 may be sized to work with existing guidewires, guide catheters, and balloon catheters.

FIG. 6A shows the example catheter caliper 210 in a starting position. In this view, the inner tube 211 is arranged within the outer tube 209 such that the markers 218A, 218B are aligned with one another. In this position, the pointer 266 is arranged at a first of the markers 264, which indicates a zero position. That is, the distance between the markers 218A, 218B is zero in this position.

FIG. 6B shows the example catheter caliper 210 in an extended or measuring position. After the physician or other user has inserted the catheter caliper 210 and positioned the distal end 214 past the blockage 154 or other artifact in the patient 150 (shown in FIG. 4), the physician or user then moves the pointer 266 along the scale 216, which moves the outer tube 209 relative to the inner tube 211 until the marker 218A is at an end of the blockage 154. That is, the marker 218B will be at a one end of the blockage 154 and the marker 218A will be at an opposite end of the blockage 154. The markers 218A, 218B are then spaced apart by a distance 270, which corresponds to the length of the blockage 154. The markers 218A, 218 B are radio-opaque so that a user can see them with medical imaging to line up the markers 218A, 218B with the blockage 154. When the markers 218A, 218B are positioned along the blockage 154, pointer 266 points at a length marker 264 that corresponds to the distance 270. In other words, the physician or user can measure how far apart the markers 218A, 218B are within the patient 150 via the scale 216 that is outside the body of the patient 150 to determine the length of the blockage.

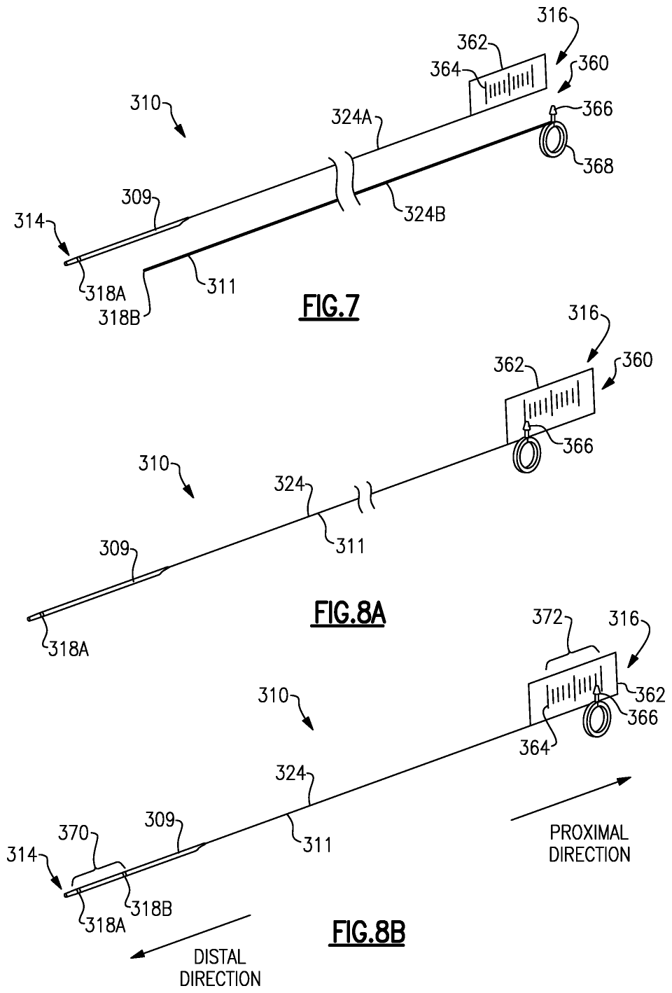

FIG. 7 shows another example catheter caliper. This catheter caliper 310 is also a two-piece device, formed from an outer tube 309 and an inner wire 311. To the extent not otherwise described or shown, the catheter caliper 310 corresponds to the catheter caliper 210 of FIGS. 5-6B, with like parts having reference numerals preappended with a “3.” The catheter caliper 310 operates in the same manner as the catheter caliper 210. The outer tube 309 has a marker 318A and the inner wire 311 has a marker 318B. A distance between the markers 318A, 318B is determinable by a scale 360 at the proximal end 316.

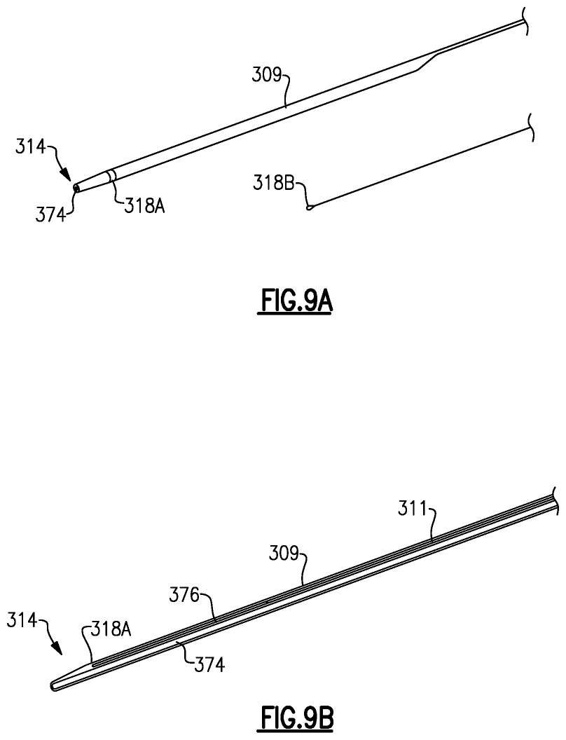

FIG. 8A shows the example catheter caliper 310 in a starting position. In this position, the inner wire is within the outer tube 309 such that the markers 318A, 318B are aligned with one another. The pointer 366 is at the zero position in the starting position.

FIG. 8B shows the example catheter caliper 310 in an extended or measuring position. After the catheter caliper 310 has been positioned adjacent the blockage or perforation/dissection 154 in the patient 150, the physician or user can then move the inner wire 311 relative to the outer tube 309 by moving the pointer 366 until the markers 318A, 318B are at opposite ends of the blockage or perforation/dissection 154. The user can then measure the distance 370 between the markers 318A, 318B by reading the distance 372 on the scale 360.

FIG. 9A shows the distal end of the catheter caliper 310 in a disassembled state. The inner wire 311 is smaller in diameter than the outer tube 309. Although the marker 318B is shown at the end of the inner wire 311, the marker 318B may be spaced from the end of the inner wire 311, in some examples.

FIG. 9B shows a cross-sectional view of the distal end of the catheter caliper 310. The outer tube 309 has an inner lumen 374 that extends a length of the outer tube 309. The inner lumen 374 is sized such that the outer tube 309 fits over the guidewire 130. A space 376 is also formed within the outer tube 309. The space 376 fits the inner wire 311. The space 376 may terminate inward of the distal end 314 of the outer tube 309. The space 376 may terminate at a location such that the marker 318B on the inner wire 311 is aligned with the marker 318A on the outer tube 309 when the inner wire 311 is fully inserted into the space 376.

Although an angioplasty procedure is described, the example catheter calipers 10, 110, 210, 310 may be used for other medical procedures in which intra-vessel measurements could be made. Although the example catheter calipers 10, 110, 210, 310 are described as measuring blockages, as discussed above, they may also be used to measure other distances, such as the length of an artery or the distance between side branches of an artery.

Known methods of estimating the size of a blockage or perforation/dissection may include using a guidewire with markers or making a digital measurement using imaging software. The disclosed catheter caliper allows a physician or other user to obtain an accurate length measurement with the guide wire of their choice. The catheter caliper 10, 110, 210, 310 provides a simple, accurate, and intuitive way to make intra-vessel measurements, which may help prevent choosing a balloon or stent that is too short or too long.

Although the different examples have the specific components shown in the illustrations, embodiments of this disclosure are not limited to those particular combinations. It is possible to use some of the components or features from one of the examples in combination with features or components from another one of the examples.

Although an embodiment of this disclosure has been explained, a worker of ordinary skill in this art would recognize that certain modifications would come within the spirit and scope of this invention.

1. A catheter caliper assembly, comprising:
a catheter caliper, including
a tube having a plurality of radiopaque markers each spaced apart by predetermined intervals; and
a wire extending from the tube, the catheter caliper defined between a distal end of the tube and a proximal end of the wire, wherein the catheter caliper is configured to be received in a vasculature of a patient.

2. The assembly of claim 1, wherein the tube is configured to receive a guidewire.

3. The assembly of claim 1, further comprising a guide catheter, wherein the catheter caliper is configured to be received in a lumen of the guide catheter.

4. A catheter caliper assembly, comprising:
a catheter caliper, including
a first component including an outer tube and a first wire extending from the outer tube, the outer tube having a first marker, the first component defined between a distal end of the outer tube and a proximal end of the first wire;
a second component configured to be received in the outer tube, and defined between a proximal end and a distal end, the distal end having a second marker;
a ruler arranged at a proximal end of one of the first and second components, the ruler having a plurality of markings each spaced apart by predetermined intervals; and
a pointer arranged at a proximal end of the other of the first and second components, the pointer configured to point to the markings on the ruler.

5. The assembly of claim 4, wherein the pointer is arranged on a finger loop or handle.

6. The assembly of claim 4, wherein moving the pointer with respect to the ruler moves one of the first marker and the second marker with respect to the other of the first and second markers.

7. The assembly of claim 4, wherein moving the first marker with respect to the second marker moves the pointer with respect to the ruler.

8. The assembly of claim 4, wherein the second component includes a second wire at the proximal end.

9. The assembly of claim 8, wherein the second component includes an inner tube at the distal end, the inner tube configured to be received in the outer tube, the inner tube including the second marker.

10. The assembly of claim 4, wherein the outer tube includes a lumen therethrough configured to receive a guidewire.

11. The assembly of claim 4, wherein the outer tube includes a space configured to receive the distal end of the second component.

12. The assembly of claim 11, wherein the space is configured such that when the distal end of the second component is received at a terminus of the space, the first and second markers are aligned.

13. A method of making intravascular measurements, the method including:
inserting a catheter caliper into a vasculature of a patient to an artifact or feature to be measured, the catheter caliper including
a tube having a plurality of radiopaque markers each spaced apart by predetermined intervals, and
a wire extending from the tube, the catheter caliper defined between a distal end of the tube and a proximal end of the wire; and
measuring the length of the artifact or feature by comparing the length of the artifact or feature to the markers on the tube.

14. The method of claim 13, wherein the artifact is a blockage, perforation, or dissection.

15. The method of claim 13, wherein the inserting includes inserting the catheter caliper over a guidewire such that the guidewire is received in the tube.

16. The method of claim 13, wherein the inserting includes inserting the catheter caliper through a guide catheter.

17. A method of making intravascular measurements, the method including:
inserting a catheter caliper into a vasculature of a patient to a location near an artifact or feature to be measured, the catheter caliper including
a first component including an outer tube and a first wire extending from the outer tube, the outer tube having a first marker, the first component defined between a distal end of the outer tube and a proximal end of the first wire;
a second component configured to be received in the outer tube, and defined between a proximal end and a distal end, the distal end having a second marker;
a ruler arranged at a proximal end of one of the first and second components, the ruler having a plurality of markings each spaced apart by predetermined intervals, and
a pointer arranged at a proximal end of the other of the first and second components, the pointer configured to point to the markings on the ruler;
positioning the distal end of the first and second components past the artifact or feature; and
moving the pointer with respect to the ruler such that the first marker moves with respect to the second marker and the first marker is positioned at a first end of the artifact or feature and the second marker is positioned at a second end of the artifact or feature.

18. The method of claim 17, wherein the second component includes an inner tube at the distal end, the inner tube configured to be received in the outer tube, the inner tube including the second marker.

19. The method of claim 17, wherein the outer tube includes a lumen therethrough configured to receive a guidewire.

20. The method of claim 17, wherein the outer tube includes a space configured to receive the distal end of the second component.
